(19) United States
(12) Patent Application Publication
(10) Pub. No.: US 2011/0124644 A1
(43) Pub. Date: May 26, 2011

(54) METHODS OF DIAGNOSING AND CHARACTERIZING CANNABINOID SIGNALING IN CROHN'S DISEASE

(75) Inventors: Stephan R. Targan, Santa Monica, CA (US); Jerome I. Rotter, Los Angeles, CA (US); Kent D. Taylor, Ventura, CA (US); David Ziring, Los Angeles, CA (US); Jonathan Braun, Tarzana, CA (US); Bo Wei, Rancho Palos Verdes, CA (US)

(73) Assignees: CEDARS-SINAI MEDICAL CENTER, Los Angeles, CA (US); THE REGENTS OF THE UNIVERSITY OF CALIFORNIA, Oakland, CA (US)

(21) Appl. No.: 12/993,685

(22) PCT Filed: May 20, 2009
(86) PCT No.: PCT/US2009/044720

§ 371 Date: Nov. 19, 2010

Related U.S. Application Data
(60) Provisional application No. 61/054,606, filed on May 20, 2008.

(51) Int. Cl. A61K 31/5377; C12Q 1/68; A61P 1/00

(52) U.S. Cl. 514/235.2; 435/6.1

(57) ABSTRACT

Diagnosis of Crohn's Disease by determining the presence or absence of variants at the CNR2 (cannabinoid) genetic locus and serological markers is disclosed Methods of diagnosing a Crohn's Disease sub-type by determining the presence or absence of one or more risk variants at the CNR2 5 locus and the presence or absence of ASCA, OmpC, 12 and/or anti-Cbir1 antibodies are included

### GOVERNMENT RIGHTS

This invention was made with U.S. Government support on behalf of the National Institutes of Health (NIH) by NIH Grant No. DK046763, NIH CHRCDA 5 K12 HD034610, and CA16042. The U.S. Government may have certain rights in this invention.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet. 29, 223 (2001); V. D. Peltekova et al., Nat Genet. 36, 471 (2004)). CD and UC are thought to be related disorders that share some genetic susceptibility loci but differ at others.

CNR2 codes for cannabinoid receptor 2 (CB2), a O-protein-coupled receptor for cannabinoids which is expressed in immune cells and involved in the development of the Th1/Th2 immune response. Serum expression of different antibodies to microbial antigens may be related to different pathophysiological mechanisms of Crohn's disease (CD). Because CD is largely an immune mediated disease, there is a need in the art to explore the relationship between CNR2 variation and sero-reactivity to microbial antigens in CD patients, as well as identify additional genetic variants and markers for IBD that may be used to define disease subtypes.

### SUMMARY OF THE INVENTION

Various embodiments include a method of diagnosing a Crohn's Disease subtype in an individual, comprising determining the presence or absence of a risk haplotype at the CNR2 genetic locus and determining the presence or absence of a high expression of ASCA antibody relative to a normal subject, where the presence of the risk haplotype at the CNR2 genetic locus and the presence of the high expression of ASCA antibody relative to a normal subject in the individual is indicative of the Crohn's Disease subtype. In another embodiment, the risk haplotype at the CNR2 genetic locus comprises Block 1 Haplotype 1. In another embodiment, the risk haplotype at the CNR2 genetic locus comprises SEQ. ID. NO.: 1, SEQ. ID, NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, or a combination thereof. In another embodiment, the high expression of ASCA antibody relative to a normal subject comprises a median antibody titer level above 0.1. In another embodiment, the individual is non-Jewish.

Other embodiments include a method of diagnosing a Crohn's Disease subtype in an individual, comprising determining the presence or absence of Block 1 Haplotype 2 at the CNR2 genetic locus and determining the presence or absence of a low expression of ASCA and/or Cbir1 antibodies relative to a normal subject, where the presence of Block 1 Haplotype 2 at the CNR2 genetic locus and the presence of the low expression of ASCA and/or Cbir1 antibodies relative to a normal subject in the individual is indicative of the Crohn's Disease subtype. In another embodiment, the low expression of ASCA antibody relative to a normal subject comprises a median antibody titer level of less than 0.5. In another embodiment, Block 1 Haplotype 2 at the CNR2 genetic locus comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, or a combination thereof. In another embodiment, the individual is non-Jewish.

Other embodiments include a method of diagnosing a Crohn's Disease subtype in an individual, comprising determining the presence or absence of Block 2 Haplotype 3 at the CNR2 genetic locus and determining the presence or absence of a low expression of OmpC and/or I2 antibodies relative to a normal subject where the presence of Block 2 haplotype 3 at the CNR2 genetic locus and determining the presence of the low expression of OmpC and/or I2 antibodies relative to a normal subject is indicative of the Crohn's Disease subtype in the individual. In another embodiment, the low expression of OmpC antibody relative to a normal subject comprises a median antibody titer level of less than 18.5. In another embodiment, the low expression of I2 antibody relative to a normal subject comprises a median antibody titer level of less than 26.0. In another embodiment, Block 2 Haplotype 3 at the CNR2 genetic locus comprises SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, or a combination thereof.

Various embodiments include a method of defining a CNR2 signaling phenotype in an individual, comprising determining the presence or absence of one or more risk haplotypes at the CNR2 locus determining the presence or absence of one or more risk serological markers, and defining the CNR2 signaling phenotype based upon the presence of one or more risk haplotypes at the CNR2 locus and the presence of one or more risk serological markers. In another embodiment, the one or more risk haplotypes at the CNR2 locus comprises Block 2 Haplotype 3. In another embodiment, the one or more risk serological markers comprises OmpC and/or I2 antibodies. In another embodiment, the CNR2 signaling phenotype comprises approximately half the level of CB2 signaling as compared to a wild type phenotype. In another embodiment, the one or more risk haplotypes at the CNR2 locus comprises Block 1 Haplotype 1 and/or Block 1 Haplotype 2. In another embodiment, the one or more risk serological markers comprises ASCA and/or Cbir antibodies. In another embodiment, the individual is non-Jewish.

Other embodiments include a method of treating Crohn's Disease in an individual, comprising determining hypofunctioning CB2 signaling based upon the presence of Block 2 Haplotype 3, OmpC antibodies, and/or I2 antibodies in a sample taken from the individual, and treating the Crohn's Disease in the individual.

Other embodiments include a method of diagnosing susceptibility to Crohn's Disease in an individual, comprising determining the presence or absence of a risk haplotype at the CNR2 genetic locus, and determining the presence or absence of a risk serological marker, where the presence of the risk haplotype at the CNR2 genetic locus and the presence of the risk serological marker in the individual is indicative of susceptibility to Crohn's Disease. In another embodiment, the risk serological marker comprises ASCA and/or Cbir antibodies. In another embodiment, the risk serological marker comprises OmpC and/or I2 antibodies.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1 depicts, in accordance with an embodiment herein, the haplotype structure of the CNR2 gene, with “1” representing the major allele, and “2” representing the “minor” allele.

FIG. 2 depicts, in accordance with an embodiment herein, results for non-Jewish subjects where B1H1 positively associated with the ASCA level, while B1H2 is negatively associated with the ASCA level.

FIG. 3 depicts, in accordance with an embodiment herein, results for non-Jewish subjects where B1H2 is negatively associated with anti-Cbir1 expression.

FIG. 4 depicts, in accordance with an embodiment herein, results for non-Jewish subjects where B2H3 was associated with lower levels of both anti-OmpC and anti-I2.

FIG. 5 depicts, in accordance with an embodiment herein, results for non-Jewish subjects where B2H3 was associated with lower levels of both anti-OmpC and anti-I2 using the sum of the quartile levels.

FIG. 6 (a)-(c) depicts, in accordance with an embodiment herein, CB2 receptor protein expression, with (a) isotype control, (b) Anti-CB2 (surface), and (c) Anti-CB2 (intracellular). 1×10⁶ cells that had either been fixed (1.6% paraformaldehyde) and permeabilized (ice cold methanol), or kept in their native state in flow staining buffer were incubated with either mouse anti-human CB2 receptor (R and D Systems, Minneapolis, Minn.), mouse anti-human CD3 (isotype control), secondary FITC goat anti-mouse only, or unstained, per well in a 96 well plate. After 30 minute incubation on ice, cells were washed and stained with FITC secondary. They were then analyzed on a FACSCalibur flow cytometer (BD Biosciences). Cells that had not been permeabilized had a staining pattern that indicated a bimodal population of receptor expression. Cells that had been fixed and permeabilized prior to staining uniformly stained positive for the presence of the antibody. When cells were stained with CD3, the isotype control, they showed the absence of staining intensity. This data indicates that methanol did not merely change the surface epitope of the CB2 protein, rendering it sensitive to antibody recognition, but methanol allowed the antibody to recognize an intracellular pool of CB2 receptor.

FIG. 7 depicts, in accordance with an embodiment herein, CB2 receptor protein expression and function. Flow cytometry data derived from staining of 40 Crohn's disease patient lymphoblastoid cell lines with monoclonal CB2 receptor (IgG2A) was generated. LCLs from patients with Crohn's disease were grown in culture and plated 1×106 per well on a 96 well plate. These cells were then incubated for 20 minutes with 10 mM IBMX, a phosphodiesterase inhibitor, and washed. Cells were incubated in the presence of various concentrations of forskolin or media control with or without the addition of 250 nM AM1241. Samples were run in triplicate. The reaction was stopped after 20 minutes by centrifugation and lysis using supplied lysis buffer (R and D Systems). Plates were frozen at −20 degrees and lysates assayed the following day for cAMP concentration using a commercial ELISA kit (R and D) and microwell ELISA plate reader. As depicted in FIG. 7, adenylate cyclase-dependent cAMP concentration obtained from the stimulated cells was proportional to the forskolin concentration, and CB2 specific agonist inhibited this activity by approximately 55% (inhibition of 25 nM forskolin-stimulated response using 250 nM AM1241).

FIG. 8 (a)-(c) depicts, in accordance with an embodiment herein, Crohn's Disease patient LCLs are heterogeneous for CB2 receptor protein expression and function. (a) The panel plots CB2 expression as measured by mean fluorescent intensity (MFI) amongst the 40 samples tested. The range of MFI is nearly 500-fold. (b) The panel depicts the CB2 receptor function as measured by intracellular cAMP level in response to CB2-specific agonist amongst the 40 patient LCL samples. (c) A linear regression analysis showed that CB2 surface receptor expression is significantly associated with function as measured by intracellular cAMP, although this association is quite weak. These results indicated that there is an effect of receptor expression on downstream function. (r2=0.1, p=0.05).

FIG. 9 (a)-(b) depicts, in accordance with an embodiment herein, CNR2 haplotype does not contribute to CB2 protein expression on IBD patient LCLs, but does contribute to receptor function. Mean fluorescent intensities for CB2 receptor staining by flow cytometry were compared between OmpC/I2+CD patients with the CNR2 SNP-associated haplotypes and those with the wild-type alleles. (a) There was no significant difference detected between groups. CB2 receptor function was also compared between OmpC/I2+ CD patient CNR2 haplotype defined subgroups using ELISA-determined intracellular cAMP concentration after stimulation with CB2-specific ligand. (b) Cells from haplotype+ CD patients had nearly half the level of CB2 signaling, indicating that this subset has an aberration in CB2 function (p=0.17).

FIG. 10 (a)-(b) depicts, in accordance with an embodiment herein, the effect of CB2 agonist on murine model of immune colitis. Splenocytes were isolated from a Gαi2−/− mouse that was sick with colitis. 1×10⁶ of these CD4 T cells (sorted using magnetic beads) were injected intraperitoneally into each of two groups of 6 Rag1 −/− mice. These mice were injected IP with either CB2 agonist (AM1241) or vehicle control. (a) Weight loss of mice treated with CB2 agonist (AM1241) compared to those treated with vehicle control (DMSO). (b) Histologic scores were determined on intestinal sections from mice sacrificed 25 days after T cell transfer (10% weight loss). This experiment was repeated on separate occasions and this figure represents the combined results of experiments involving 24 mice.

FIG. 11 (a) depicts, in accordance with an embodiment herein, the effect of CB2 agonist on murine model of acute colitis. Groups of 5 wildtype C3H/HeJ mice were challenged with either DSS 3.5% or acidified water and injected intraperitoneally with CB2 agonist (AM1241(AM)) or antagonist (SR144928 (SR)) or vehicle control (DMSO (D) for AM1241, or EtOH (E) for SR144928) and monitored for weight loss. Intestinal sections were scored histologically at day 10. (b) and (c.) depicts the comparison of DSS colitis in CB2−/− and WT mice, with (b) weight loss and (c) histologic scoring. This is representative of three or more independent experiments.

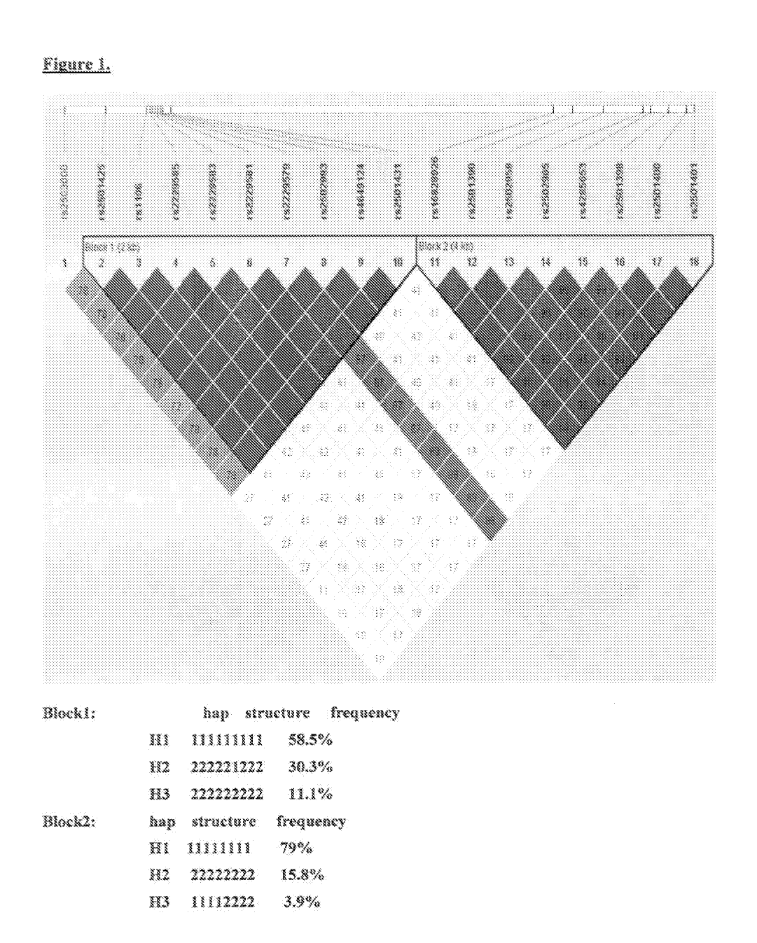

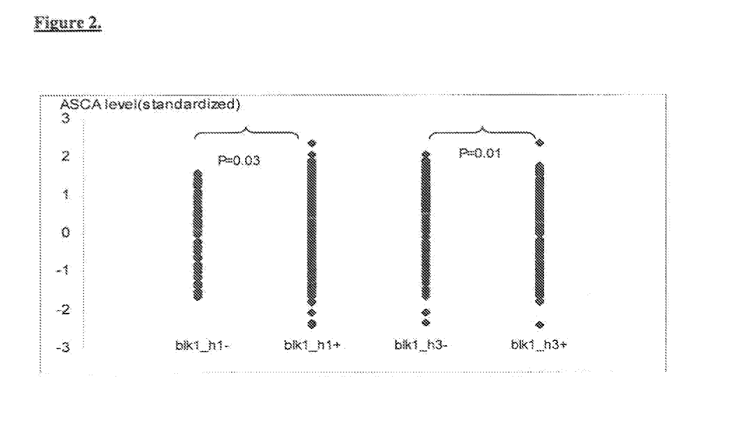

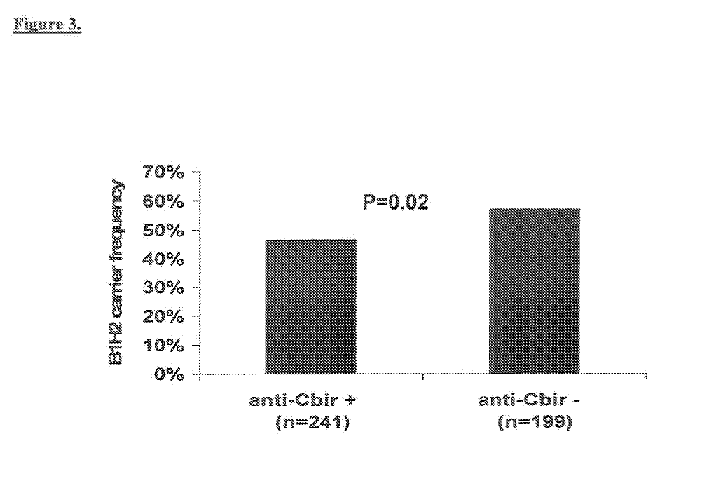

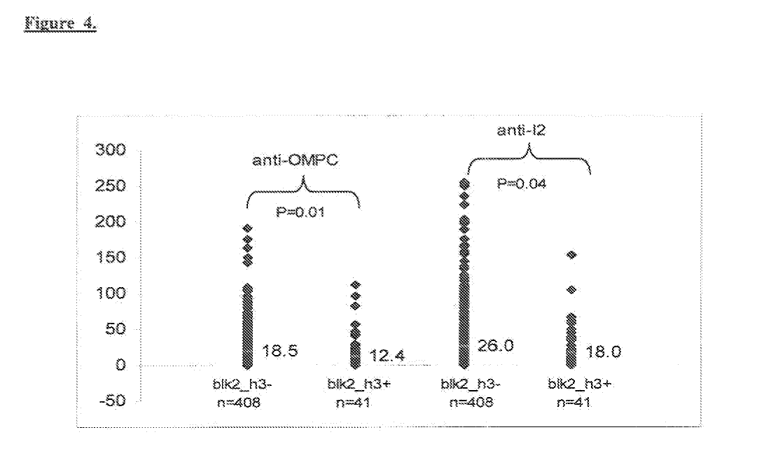

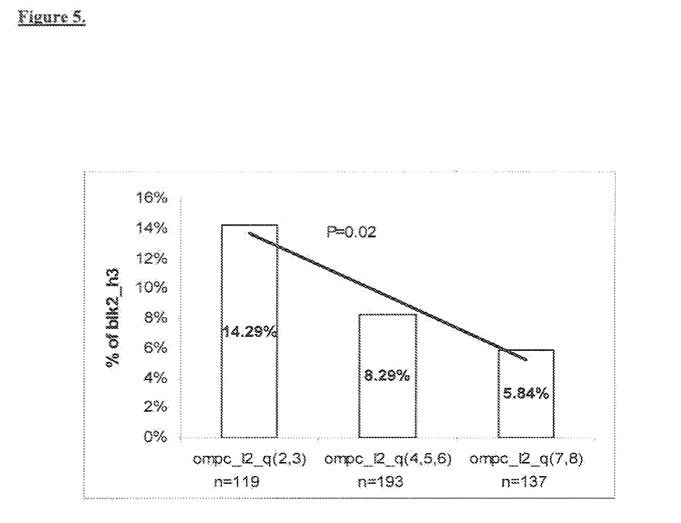

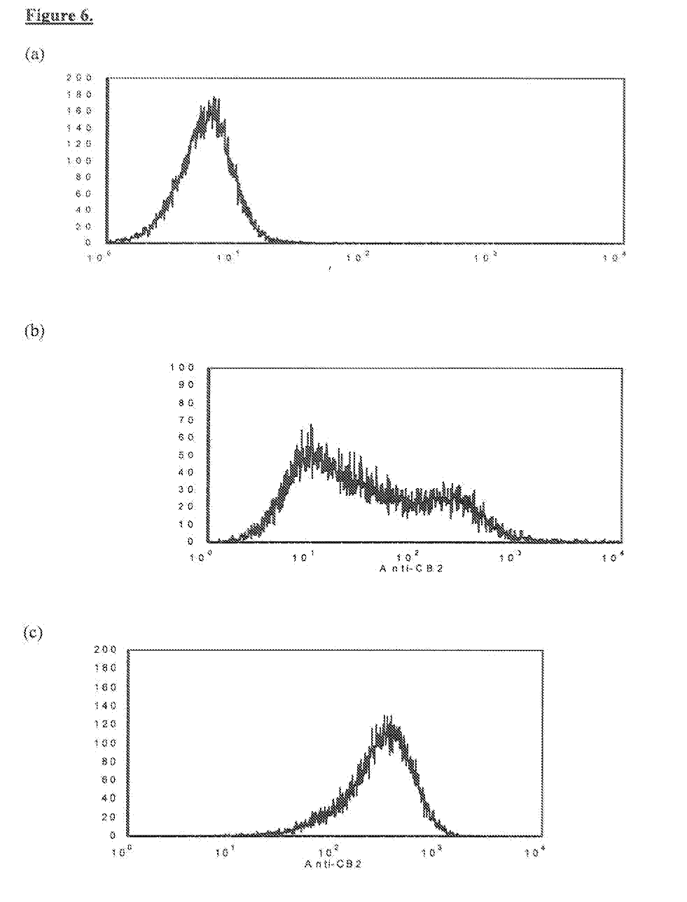

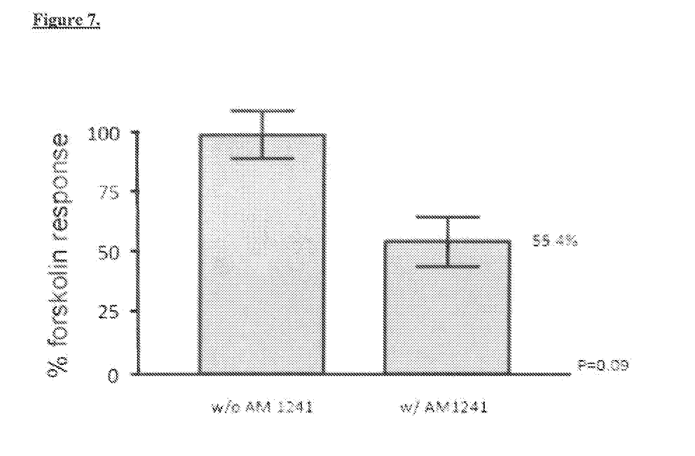

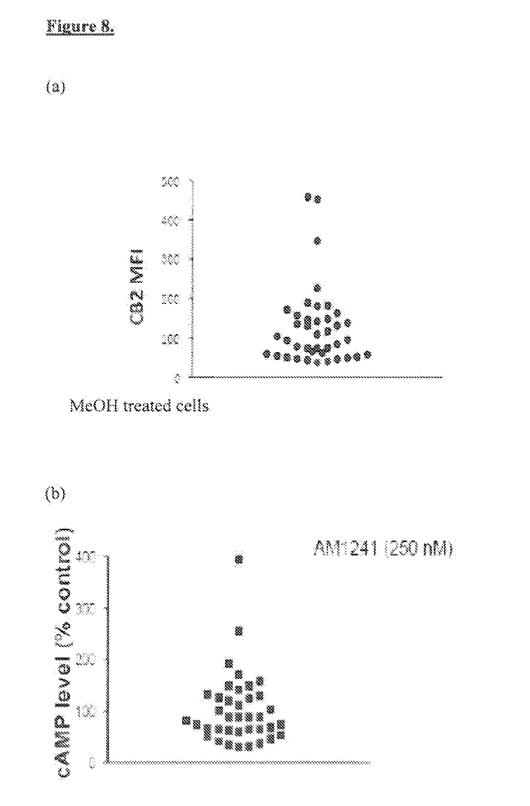

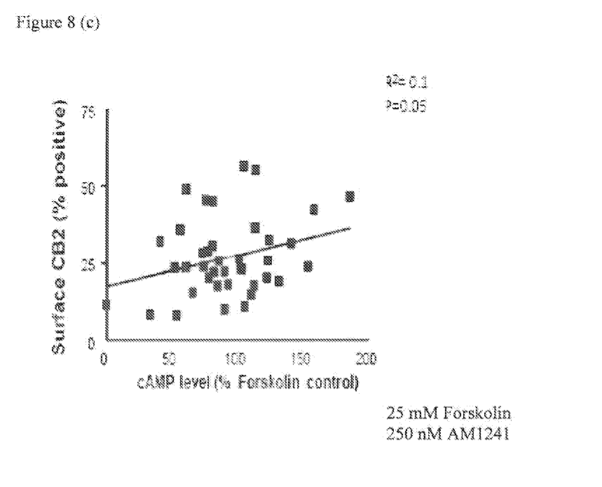

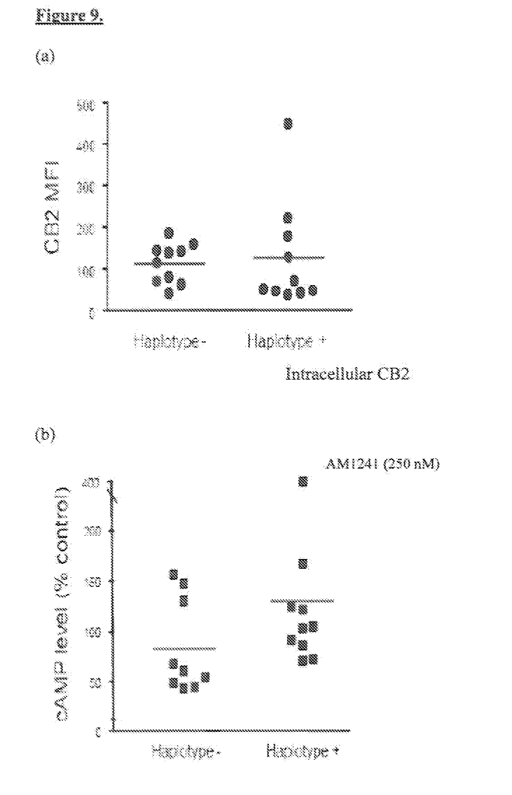

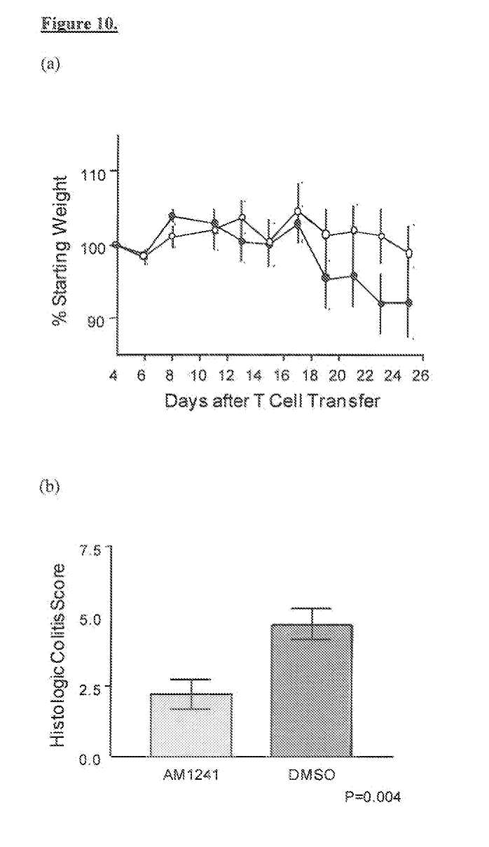

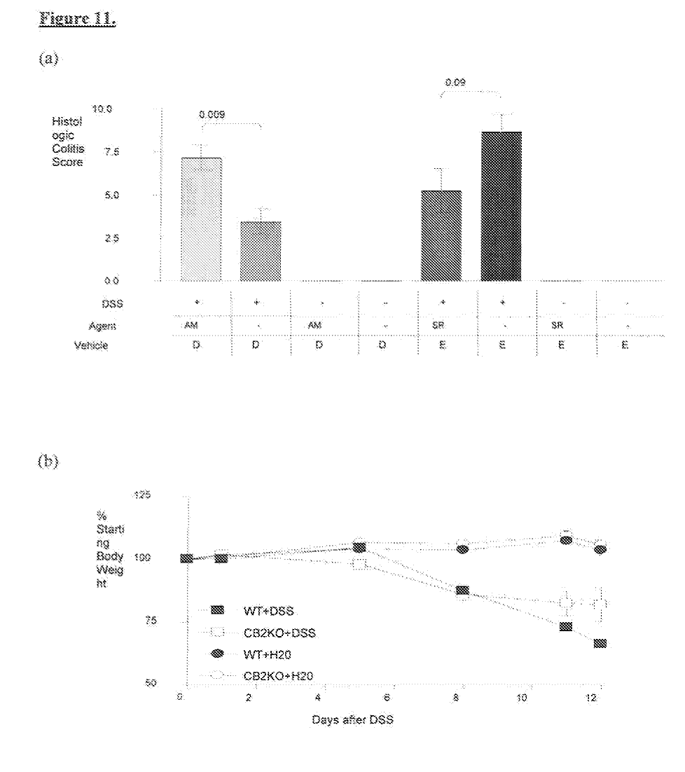

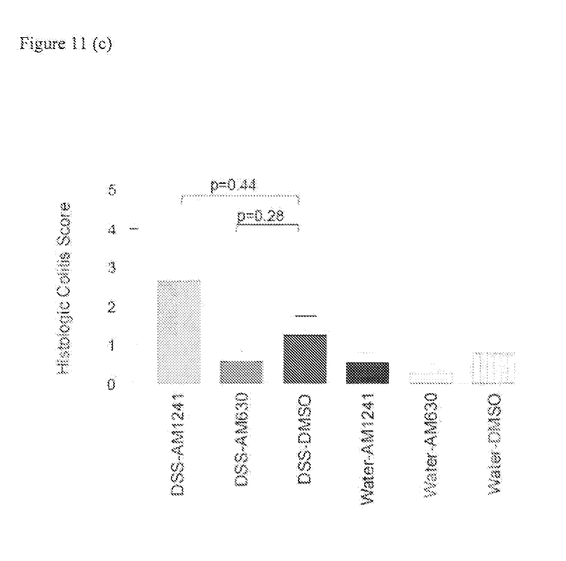

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Protective” and “protection” as used herein refer to a decrease in susceptibility to IBD, including but not limited to CD and UC.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, the terms “B1H1”, “B1H2”, “B1H3”, “B2H1”, “B2H2” and “B2H3” mean Block 1 haplotype 1, Block 1 haplotype 2, Block 1 haplotype 3, Block 2 haplotype 1, Block 2 haplotype 2, Block 2 haplotype 3, respectively, at the CNR2 genetic locus. An example of the CNR2 genetic locus is included herein as SEQ. ID. NO.: 18.

As used herein, Block 1 of the CNR2 genetic locus includes rs2501425, rs1106, rs2229585, rs2229583, rs2229581, rs2229579, rs2502993, rs4649124, rs2501431, which are described herein as SEQ. ID. NO.: 1, SEQ, ID. NO.: 2, SEQ, ID. NO.: 3, SEQ. ID. NO.: 4, SEQ. ID. NO.: 5, SEQ. ID. NO.: 6, SEQ. ID. NO.: 7, SEQ. ID. NO.: 8, and SEQ. ID. NO.: 9, respectively. Block 2 of the CNR2 genetic locus includes rs16828926, rs2501390, rs2502959, rs2502965, rs4285653, rs2501398, rs2501400, rs2501401, which are described herein as SEQ. ID. NO.: 10, SEQ. ID. NO.: 11, SEQ. ID. NO.: 12, SEQ. ID. NO.: 13, SEQ. ID. NO.: 14, SEQ. ID, NO.: 15, SEQ. ID. NO.: 16, and SEQ. ID. NO.: 17, respectively. The aforementioned sequences are examples of tagSNPs which may be used to define Block 1 and Block 2 of the CNR2 genetic locus, respectively.

As used herein, the term “CNR2” is an abbreviation for the genetic locus that codes for cannabinoid receptor 2 (“CB2”), a G-protein coupled receptor for cannabinoids.

As used herein, the term “SNP” refers to single nucleotide polymorphisms.

As used herein, the term “CD” refers to Crohn's Disease.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors evaluated the relationship between CNR2 variation and sero-reactivity to microbial antigens in CD patients. 763 CD (314 Jews) were genotyped for 18 CNR2 tagSNPs by the Illumina GoldenGate Assay. Sera were tested for ASCA, anti-12, anti-OmpC, and anti-CBir1 by ELISA. Association between haplotypes and antibodies was tested using Chi-square and Wilcoxin Rank. Because anti-OmpC and anti-I2 had similar reactive profiles, the inventors grouped these two antibodies using quartile sum (QS) for additional analysis. Mantel-Haenszel chi-square was performed for trend.

As further disclosed herein, two haplotype blocks were observed. 1) In non-Jews, block 1 haplotype 1 (B1H1) was positively associated with ASCA expression (median level, B1H1+: 0.4 vs. B1H1−: 0.1, p=0.03); while block 1 haplotype 2 (B1H2) was negatively associated with ASCA expression (median level, B1H2+: 0.3 vs. B1H2−: 0.5, p=0.01). B1H2 was also negatively associated with anti-CBir1 expression (46.5% of CD subjects with B1H2 were anti-CBir1+ vs. 57.3% of CD subjects with B1H2 were anti-CBir1−, p=0.02). 2). In non-Jews, block 2 haplotype 3 (B2H3) was associated with lower expression of both anti-OmpC (median level, B2H3+: 12.4 vs. B2H3−: 18.5, p=0.01) and anti-I2 (median level, B2H3+: 18.0 vs. B2H3−: 26.0, p=0.04). This association of B2H3 with lower antibody expression was also observed using the sum of the quartiles of expression (QS 2 & 3: 14.3% of CD subjects had B2H3, QS 4-6: 8.3%, QS 7 & 8: 5.8%, p trend=0.02). No significant association was observed in the Jewish subgroup. In one embodiment, the invention provides a method of diagnosing a Crohn's Disease subtype in an individual by determining the presence or absence of a risk haplotype at the CNR2 locus and the presence or absence of a high magnitude of ASCA expression relative to a healthy subject, where the presence of the risk haplotype at the CNR2 locus and the presence of the high magnitude of ASCA expression relative to a healthy subject is indicative of the Crohn's Disease subtype. In another embodiment, the risk haplotype is Block 1 haplotype 1 at the CNR2 locus. In another embodiment, the present invention provides a method of treating Crohn's Disease in an individual by determining the presence of the Block 1 haplotype 1 at the CNR2 locus and/or the presence of the high magnitude of ASCA expression relative to a healthy subject and treating the Crohn's Disease. In another embodiment, the individual is non-Jewish.

In another embodiment, the invention provides a method of diagnosing a Crohn's Disease subtype in an individual by determining the presence or absence of Block 1 haplotype 2 at the CNR2 locus and the presence or absence of a high magnitude of ASCA and/or Cbir1 expression relative to a healthy subject, where the presence of Block 1 haplotype 2 at the CNR2 locus and the absence of a high magnitude of ASCA and/or Cbir1 expression relative to a healthy subject is indicative of the Crohn's Disease subtype. In another embodiment, the present invention provides a method of treating Crohn's Disease in an individual by determining the presence of the Block 1 haplotype 2 at the CNR2 locus and/or the absence of a high magnitude of ASCA and Cbir1 expression relative to a healthy individual and treating the Crohn's Disease. In another embodiment, the individual is non-Jewish.

In another embodiment, the invention provides a method of diagnosing a Crohn's Disease subtype in an individual by determining the presence or absence of Block 2 haplotype 3 at the CNR2 locus and the presence or absence of a high magnitude of OmpC and/or I2 expression relative to a healthy subject, where the presence of Block 2 haplotype 3 at the CNR2 locus and the absence of a high magnitude of OmpC and/or I2 expression relative to a healthy subject is indicative of the Crohn's Disease subtype. In another embodiment, the present invention provides a method of treating Crohn's Disease in an individual by determining the presence of the Block 2 haplotype 3 at the CNR2 locus and/or the absence of a high magnitude of OmpC and I2 expression relative to a healthy subject and treating the Crohn's Disease. In another embodiment, the individual is non-Jewish.

In another embodiment, the present invention provides a method of defining a CNR2 signaling phenotype in an individual by determining the presence or absence of one or more risk haplotypes at the CNR2 locus, determining the presence or absence of one or more risk serological markers, and defining the CNR2 signaling phenotype based upon the presence of one or more risk haplotypes at the CNR2 locus and the presence of one or more risk serological markers. In another embodiment, the risk haplotype at the CNR2 locus is Block 2 Haplotype 3 and the risk serological marker is OmpC and/or I2 antibodies, where the presence of Block 2 Haplotype 3 and the presence of OmpC and/or I2 antibodies is indicative of a hypofunctional CNR2 allele. In another embodiment, the risk haplotype at the CNR2 locus includes Block 1 haplotype 1 and/or block 1 haplotype 2. In another embodiment, the risk serological markers include ASCA and/or Cbir antibodies. In another embodiment, the individual is non-Jewish.

As readily apparent to one of skill in the art, the ability to diagnose a disease subtype, such as a Crohn's Disease subtype, has several possible applications and enormous value. For example, in the instance of a clinical trial for a possible Crohn's Disease treatment, the ability to determine whether a candidate treatment would be effective or ineffective for a specific subgroup of possible subjects, would both save enormous cost as well as increase the overall effectiveness of the trial. Also, for example, having the ability to diagnose a Crohn's Disease subgroup would allow personalized and more effective treatment for a patient, where a specific treatment regimen may be designed and administered dependent upon which Crohn's Disease subgroup the patient has been diagnosed with. Similarly, a method of defining a CNR2 signaling phenotype in an individual with Crohn's Disease has great value and many possible applications. For example, the ability to determine that a subset of patients with Crohn's Disease or inflammatory bowel disease have a specific functionality of CNR2 signaling enables the prediction of patient response to CB2 targeted therapy, as well as enable the development of pharmaceuticals and treatments that are specifically tailored for the patient.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gcl compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### CNR2 Association with Crohn's Disease and Sero-Reactivity

CNR2 codes for cannabinoid receptor 2 (CB2), a G-protein-coupled receptor for cannabinoids. CB2 is expressed in immune cells and is involved in the development of the Th1/Th2 immune response, and resistance to experimental colitis. Serum expression of different antibodies to microbial antigens may be related to different pathophysiological mechanisms of Crohn's disease (CD). The inventors evaluated the relationship between CNR2 variation and sero-reactivity to microbial antigens in CD patients. 763 CD (314 Jews) were genotyped for 18 CNR2 tagSNPs by the Illumina GoldenGate Assay. Sera were tested for ASCA, anti-OmpC, and anti-CBir1 by ELISA. Association between haplotypes and antibodies was tested using Chi-square and Wilcoxin Rank. Because anti-OmpC and anti-I2 had similar reactive profiles, the inventors grouped these two antibodies using quartile sum (QS) for additional analysis. Mantel-Haenszel chi-square was performed for trend.

Two haplotype blocks were observed. 1) In non-Jews, block 1 haplotype 1 (B1H1) was positively associated with ASCA expression (median level, B1H1+: 0.4 vs. B1H1−: 0.1, p=0.03); while block 1 haplotype 2 (B1H2) was negatively associated with ASCA expression (median level, B1H2+: 0.3 vs. B1H2−: 0.5, p=0.01). B1H2 was also negatively associated with anti-CBir1 expression (46.5% of CD subjects with B1H2 were anti-CBir1+ vs. 57.3% of CD subjects with B1H2 were anti-CBir1−, p=0.02). 2). In non-Jews, block 2 haplotype 3 (B2H3) was associated with lower expression of both anti-OmpC (median level, B2H3+: 12.4 vs. B2H3−: 18.5, p=0.01) and anti-I2 (median level, B2H3+: 18.0 vs. B2H3−: 26.0, p=0.04). This association of B2H3 with lower antibody expression was also observed using the sum of the quartiles of expression (QS 2 & 3: 14.3% of CD subjects had B2H3, QS 4-6: 8.3%, QS 7 & 8: 5.8%, p trend=0.02). No significant association was observed in the Jewish subgroup.

### Example 2

### CNR2 Association with Crohn's Disease and Sera-Reactivity

### Methods

18 CNR2 tag SNPs were tested in 763 CD patients (314 Jews). Sera were analyzed for expression of ASCA, anti-I2, anti-OmpC and anti-CBir by ELISA. Haplotype blocks were determined by Haploview and individual haplotypes were assigned by PHASE. Quartile sum (QS) of anti-OmpC and anti-I2 were calculated by assigning each antibody a quartile score according to its distribution (1, 2, 3, 4) and summing the individual quartiles. Association between haplotypes and antibodies was tested using Chi-square and Wilcoxon Rank. Mantel-Haenszel chi-square was performed for the trend test. All analyses were done in the total sample first, and then separately in Jewish and non-Jewish subjects.

### Example 3

### CNR2 Association with Crohn's Disease and Sero-Reactivity

### Results

In non-Jews, block 1 haplotype 1 (B1H1) was positively associated with ASCA level, while B1H2 was negatively associated with ASCA level. In non-Jews, B1H2 was also negatively associated with anti-CBir1 expression. In non-Jews, B2H3 was associated with lower level of both anti-OmpC and anti-I2. This association of B2H3 with lower antibody expression was also observed using the sum of the quartile levels. No significant association was observed in the Jewish subgroup.

CNR2 SNP-determined haplotypes are found to be associated with the presence of distinct antimicrobial serologies, serologic level, and antibody quartile sums.

### Example 4

### CNR2 Associated with Crohn's Disease

CB2, the peripheral cannabinoid receptor, is largely expressed by immune cells and is required for the development of key immunoregulatory subsets, and CB2 agonists are effective in IBD mouse models and some clinical studies of IBD therapy. As disclosed herein, CNR2, the gene encoding the human CB2 receptor, demonstrated haplotype variation in patients with Crohn's disease (CD), and that these changes had consequences with regard to expression and immune cell function.

A CNR2 candidate gene case-control analysis was performed with 449 patients with Crohn's disease, 215 with ulcerative colitis, and 201 controls. Using data from the International HapMap Consortium, CNR2 tagged single nucleotide polymorphisms (SNPs) were chosen to tag major Caucasian haplotypes. The Illumina Golden Gate oligonucleotide ligation assay was used to genotype the tags. Haplotypes were assigned using PHASE v2 with greater than 95% confidence. EBV immortalized lymphoblastoid cell lines (LCLs) were analyzed for CB2 receptor by flow cytometry, and for CB2 receptor function (Gi-mediated adenylate cyclase inhibition) by CB2 agonist (AM1241) inhibition of forskolin-stimulated cAMP production, measured by ELISA.

In non-Jewish CD patients, CNR2 haplotypes protective for disease also had a lower anti-microbial antibody quartile sum. Data on protein expression for specific CNR2 haplotypes as measured by flow cytometry and CB2 receptor function as measured by cAMP in forskolin-stimulated LCLs in the presence of AM1241 are further disclosed herein.

CNR2 assigned SNP haplotypes inversely correlated with anti-microbial antibody quartile sums in non-Jewish patients with Crohn's disease. CNR2 is an IBD-associated gene. Archived patient LCLs provide an advantage in studying genotype-phenotype correlation as they allow the study of both protein expression and immune cell function. Expression and signaling profiling of these archived samples showed striking heterogeneity. Thus, differences in CB2 receptor expression or function contribute to predisposition to IBD.

### Example 5

### CNR2 Associated with Crohn's Disease

### Conclusions

CNR2 haplotypes define subsets of patients with Crohn's disease, with CB2 expression and signaling varying among patients with Crohn's Disease. Haplotypes may be used to distinguish CNR2 signaling phenotypes. Some implications are that lymphoblastoid cell lines may be utilized for functional genomics, data can be acquired in 1/10 the time, sample size of 8,000 may be utilized with no blood draws, CB2 pharmacogenomics, and patient response to CB2-targeted therapy may be predicted.

### Example 6

Hypofunctional Alleles of CNR2 Associated with IBD

### Generation of LCLs

Lymphoblastoid cell lines were derived from primary human IBD patient donor lymphocytes by EBV infection according to standard protocol.

### Example 7

### Hypofunctional Alleles of CNR2 Associated with IBD

### Antibodies

Unconjugated and fluorochrome-conjugated monoclonal antibodies specific for human CD3 (IgG2A isotype control), CD 19 (flow compensation control), and mouse IgG (secondary) were obtained from BD Biosciences (San Diego, Calif.). Mouse anti-human CB2 receptor antibody was purchased from R & D Systems (Minneapolis, Minn.).

### Example 8

### Hypofunctional Alleles of CNR2 Associated with IBD

### CNR2 Candidate Gene Case-Control Analysis

A CNR2 candidate gene case-control analysis was performed with 763 patients with Crohn's disease, 215 with ulcerative colitis, and 201 controls. Using data from the International HapMap Consortium, 18 CNR2 tagged single nucleotide polymorphisms (SNPs) were chosen to tag major Caucasian haplotypes. The Illumina Golden Gate oligonucleotide ligation assay was used to genotype the tags. Haplotypes were assigned computationally using PHASE v2 (M Stephens, University of Chicago) with greater than 95% confidence. Anti-microbial serologies had been determined for each patient for specific activity against ASCA, CBir, OmpC, and I2. The association of the presence or absence of each CNR2 haplotype with each anti-microbial serologic titer was then quantified.

### Example 9

### Hypofunctional Alleles of CNR2 Associated with IBD

### CB2 Protein Expression

40 LCLs from patients with Crohn's disease who had the haplotypes associated with OmpC/I2 and ASCA/CBir, and 40 LCLs from patients without these haplotypes were thawed from the archives. Lymphoblastoid cell lines were grown in culture (37° C., 5% CO₂) in RPMI 1640 media (Gibco) with 10% added fetal calf serum and 1% penicillin/streptomycin until they had reached 2.0-2.5×10⁷ cells per T75 flask. The lymphocytes were then counted and suspended at 1×10⁶ cells/mL in culture media. Half of the aliquots were fixed with 10% paraformaldehyde, washed, and permeabilized with 100% ice cold methanol prior to staining. The other aliquots were maintained in flow staining buffer (1×PBS (GIBCO/Invitrogen, Carlsbad, Calif.) with 0.5% bovine serum albumin (BSA)). All were then stained with the appropriate antibodies (CB2, CD19, CD3 isotype control, secondary antibody only) in flow staining buffer and analyzed by flow cytometry. CB2-positive and -negative staining cells were detected in the non-permeabilized fractions by their intensity of FITC staining. Non-conjugated and fluorescence-conjugated antibodies were obtained from BD Pharmingen (San Diego, Calif.). All staining profiles were based on live gates, as determined by forward and side scatter. Data were collected on a FACSCalibur flow cytometer (BD Biosciences, San Jose, Calif.) and analyzed using FCS software.

### Example 10

### Hypofunctional Alleles of CNR2 Associated with IBD

### CB2 Signaling

To assess the LCL response to CB2 specific agonist, intracellular cAMP was quantified using a standard ELISA kit (R and D Systems). LCLs were suspended at 5×10⁵ cells/well in 100 mcL of 37° C. culture media in duplicate on a 96 well plate. 20 μM IBMX, a phosphodiesterase inhibitor was added to all the wells and the plate was incubated at 37° C. for 20 minutes. The cells were then washed and resuspended in either media alone, 25 μM forskolin, or 50 μM forskolin for 12 minutes with the addition of either 250 nM AM1241 or 0.01% DMSO vehicle control. The LCLs were then spun down, lysed using supplied lysis buffer, and frozen at −20° C. After thawing, plates were prepared according to the supplied ELISA protocol and read on a Molecular Devices Vmax microplate reader at 450 nm. A standard curve was constructed by 4 parameter logistic regression and unknown concentrations were computed using the Molecular Devices Softmax Pro software.

### Example 11

### Hypofunctional Alleles of CNR2 Associated with IBD

### Mice

C57BL/6, C3H/HeJ, and RAG1−/− (129/Sv background) were obtained from Jackson Laboratories (Bar Harbor, Me.) and housed at UCLA in specific pathogen free (SPF) conditions. CB2−/− and heterozygote littermate control mice were backcrossed on C57BL/6 more than 11 generations. CB2 deficiency was confirmed by PCR. Mice were housed with sterilized cages, bedding, rodent chow (egg-free), and water. Each room is HEPA filtered and all mice are maintained in microisolator cages that are opened only in flow hoods using sterile procedure. Unless otherwise specified, animals examined here were both male and female, from 6-8 weeks of age, and were age matched. All animal procedures were performed according to the guidelines of the UCLA Animal Care and Use Committee Research Committee.

### Example 12

### Hypofunctional Alleles of CNR2 Associated with IBD

### Acute and Immune Colitis Models

3.5% Dextran sulfate sodium (prepared in acidified drinking water) was obtained from MP Biomedicals, Inc (Solon, Ohio). Acidified drinking water of colitis-induced mice was replaced with DSS solution for a total of eight days. Controls were given standard acidified drinking water. All mice were sacrificed on day 12, at which time the DSS-treated mice had lost approximately 20% of their body weight. Experiments were performed in duplicate, with groups of 6 mice in each treatment arm. AM1241 (Sigma-Aldrich, MO, in DMSO, 1 mg/mL) was suspended in 250 μL sterile 0.9% saline and injected intraperitoneally (i.p.) at a dose of 1 mg/kg beginning two days prior to the administration of DSS (or acidified drinking water in the control group) and then every other day until the mice were euthanized. SR144528, CB2-specific antagonist (in ethanol, 5.1 mg/mL, a gift of Dr. Nancy Buckley), was suspended in 250 μL sterile 0.9% saline and administered i.p. at a dose of 1 mg/kg with the same dosing frequency. Ethanol was given i.p. in the same volume and served as a vehicle control. In an alternative set of experiments, AM630 was suspended in DMSO and administered i.p. at a dose of 1 mg/kg with the same dose schedule as that used for AM1241 and SR144528. In this case, DMSO was used as a vehicle control.

To model immune colitis, CD3-positive T cells were isolated using MACS beads (Miltenyi Biotec, Auburn, Calif.) from the homogenized single cell suspensions of spleens isolated from sick (marked by weight loss, diarrhea, and rectal prolapse at approximately 6 weeks) Gαi2−/− mice. These T cells were then suspended at a concentration of 1×10⁶ cells in 100 μL of sterile 0.9% saline. The cell suspensions were then injected via tail vein into groups of 6 Rag1 −/− recipients. These mice were then allowed 3 days to recover before being dosed with AM1241 or DMSO vehicle control i.p. every other day for 22 days at which time they were euthanized.

Colitis was quantified by both weight loss and histology of intestinal sections. At the conclusion of the studies, the mice were sacrificed using inhaled halothane and sections of cecum, proximal, and distal colon were processed for histology using hematoxylin and eosin counterstains. Each section was then scored by one blinded pathologist using a standard experimental colitis scoring system.

### Example 13

### Hypofunctional Alleles of CNR2 Associated with IBD

### Statistical Analysis

Statistical analysis was conducted, and graphs were constructed using GraphPad Prism software (murine studies, graphpad.com) and SAS (translational studies, SAS Institute, Inc.). For the murine colitis studies, we conducted a two-sided student t-test with a 95% confidence interval to determine significance of differences seen between the two groups. For the human genetic and translational studies, association between haplotypes and antibodies was tested using Chi-square and Wilcoxin Rank. Because anti-OmpC and anti-I2 had similar reactive profiles, we grouped these two antibodies using quartile sum (QS) for additional analysis. Mantel-Haenszel chi-square analysis was performed to examine trends between haplotype and phenotype. For all statistical tests, significance was defined as p<0.05.

### Example 14

### Hypofunctional Alleles of CNR2 Associated with IBD

### CNR2 SNP Haplotypes are Associated with Crohn's Disease

More than 8,000 IBD patient lymphoblastoid cell lines (LCLs) have been archived over the last 15 years at Cedars-Sinai Medical Center Inflammatory Bowel Disease Research Center. The inventors analyzed those from 763 patients with CD (314 Jews), 351 UC (136 Jews) and 251 controls (51 Jews) to determine whether certain CNR2 SNPs are overrepresented in particular IBD patient populations. These patients were matched for sex, age, disease activity, disease location, and IBD type.

Candidate gene case-control analysis revealed that CNR2 single nucleotide polymorphism (SNP)-defined haplotypes are associated with specific subsets of patients with Crohn's disease. A CNR2 SNP constructed HapMap illustrates that these haplotypes fall into two discrete blocks. These SNP blocks are arranged so that the SNPs with the highest degree of linkage disequilibrium fall together.

### Example 15

### Hypofunctional Alleles of CNR2 Associated with IBD

### Antimicrobial Serologies Help Define Crohn's Disease Patient Subtypes

The inventors found that in non-Jewish patients, block 1, haplotype 2 is negatively associated with the presence of antibodies to CBir. Block 2, haplotype 3 is also negatively associated with the presence of antibodies to I2. Not only are these haplotypes associated absolutely with the presence or absence of the antibody response, but the risk of having the haplotype is correlated (or inversely correlated) with antibody titer for OmpC and/or I2. Thus, CNR2 is a newly described IBD-associated gene.

### Example 16

### Hypofunctional Alleles of CNR2 Associated with IBD

### CNR2 SNP-Assigned Haplotypes do not Determine CB2 Expression

CNR2 haplotypes may differentially define CB2 protein expression or function. We therefore analyzed the concentration of CB2 surface receptor on our patient derived LCL samples. Anonymized de-identified LCLs were grown in culture and analyzed by flow cytometry. Surface expression was assessed by staining of intact cells with anti-CB2 antibody, revealing biphasic levels of expression. When cells were permeabilized, it was found that the MFI of the positive staining population shifted, with nearly 100% of the cells staining for the presence of the protein. This indicates that these lymphoblastoid cells contain a large intracellular reservoir of CB2 receptor. The inventors then calculated MFI of the CB2 receptor antibody signal and compared the levels from cells of patients possessing the haplotypes with those with the wild-type alleles. As described herein, there was no difference in MFI of the CB2 signal in either permeabilized or non-permeabilized cells between haplotype-positive patients and haplotype negative patients.

Control experiments confirmed that relative expression levels of both surface and total CB2 were reproducible in each LCL cell line; and, where available, were consistent between independent LCLs derived from single subjects. Therefore, the diversity of CB2 expression was a distinctive subject-associated trait that did not appear to reflect analytic issues in LCL analysis. However, the diversity of CB2 expression was not significant affected by CNR2 haplotypes.

### Example 17

### Hypofunctional Alleles of CNR2 Associated with IBD

### CNR Haplotypes are Associated with Defective CB2 Signaling in Patient LCLs

As the inventors knew that the CNR2 SNP haplotypes define IBD patient subsets genetically, we wondered whether these alleles led to differences in protein function. CB2 is a typical seven transmembrane G-inhibitory protein coupled receptor, and ligation of this receptor causes a decrease in intracellular cAMP. To test the function of the CB2 receptor, the inventors cultured IBD patient LCLs with or without forskolin in the presence or absence of CB2-specific agonist (AM1241). No statistically significant differences were found between the CB2 signaling and CNR2 haplotypes. As in the case of CB2 expression, C132 signaling levels was a consistent trait of LCLs derived from individual subjects.

The inventors next assessed the heterogeneity of CB2 expression and function in the LCLs of our patient population. The inventors found a high degree of heterogeneity among these samples. Further, a linear regression analysis showed that CB2 surface receptor expression was significantly associated with function as measured by intracellular cAMP. These results indicated that there may be an effect of receptor expression on downstream function.

### Example 18

### Hypofunctional Alleles of CNR2 Associated with IBD

### CB2 Agonist Ameliorates Immune Colitis

While CB2 agonist and antagonists do not affect immune cell formation in weanling mice, there is substantial literature that CB2 agonists can acutely induce anti-inflammatory functions of B cells and macrophages in vitro. The inventors wondered whether CB2-specific agonist could control the development of immune-mediated disease, such as inflammatory bowel disease. Because there is no reported study on CB2 pharmacotherapy in colitis, the inventors tested the action of CB2 agonist, C132 antagonist, or vehicle controls on murine models of colitis.

Gαi2−/− mice undergo spontaneous colitis predominating with TH1 and TH17-mediated inflammation typical of human Crohn's disease, and reliable temporal induction of colitis (3-6 weeks) is achieved by transfer Gαi2−/− T cells into immunodeficient recipients (Rag1 −/− mice). To determine the effect of CB2 modulation in a well-established model of Th1 colitis, groups of 12 Rag 1 −/− mice were injected intraperitoneally with either CB2 specific agonist (AM1241) or vehicle control (DMSO) beginning two days prior to the transfer of colitigenic Gαi2−/− T cells and every other day thereafter. Disease activity was assessed by body weight and monitored for the presence of disease activity by observing for bloody stools or rectal prolapse.

Mice treated with CB2 agonist lost significantly less weight than the group treated with vehicle control. To determine the relationship of this clinical finding to intestinal inflammation, mice were sacrificed 8 weeks after T cell transfer (a time at which control mice had lost 10% of their body weight), and tissues were harvested for quantitative histologic scoring of intestinal inflammation. Mice treated with CB2 agonist were dramatically reduced for inflammation compared to those receiving vehicle control.

### Example 19

### Hypofunctional Alleles of CNR2 Associated with IBD

### CB2 Agonist Exacerbates Acute Colitis

The effect of CB2-specific agonist in the setting of acute colitis was evaluated using dextran sodium sulfate (DSS), an ingested agent which induces acute, non-T cell mediated colitis (weight loss and bloody diarrhea) within three to five days. Mice were pre-treated with AM1241 or vehicle control, and then monitored for the level of acute colitis induced by DSS ingestion. Surprisingly, DSS colitis was significantly more severe in mice treated with AM1241 compared to vehicle control. Moreover, the CB2 antagonist SR144528 ameliorated DSS colitis (although the ethanol vehicle for the antagonist itself increased the severity of colitis). Therefore, this finding was reassessed using the CB2 antagonist AM630 (and its vehicle control, DMSO, which does not affect DSS colitis). Concordantly, AM630 reduced DSS colitis severity compared to vehicle control. These opposite findings in these two models of colitis indicate that the CB2 receptor system has pleiotropic effects on the cell types predominating in acute versus immune intestinal inflammation.

### Example 20

### Hypofunctional Alleles of CNR2 Associated with IBD

### CB2 Deficiency is not Associated with More Severe Acute Colitis

In order to determine whether genetic sufficiency is required for the susceptibility to acute DSS-mediated colitis, the inventors modeled this system in groups of CB2−/− and CB2 +/+ mice. Mice treated with DSS had equivalent weight loss and clinical symptomatology as well as colitis severity as scored by histology. (By days 10 and 12, more mice died in the CB2−/− group. So, the slightly favorable weight change in the CB2−/− survivors probably reflects the absence of the non-survivors.)

This finding indicates that a genetic deficiency of CB2 does not predispose the mice to acute colitis.

### Example 21

### Hypofunctional Alleles of CNR2 Associated with IBD

### Generally

Endocannabinoid sensing by the CB2 receptor regulates the immune system and inflammatory pain in health and disease, including mouse models of IBD. In order to study whether alleles of CNR2 may be associated with susceptibility in IBD, candidate gene case control analysis was performed for CNR2, the gene encoding the CB2 receptor. Non-Jewish patients with IBD (449 patients with Crohn's disease, 215 with ulcerative colitis, and 201 controls) were analyzed by tagged single nucleotide polymorphisms (SNPs). Assays of CB2 protein expression and cAMP-dependent signaling were performed on EBV immortalized lymphoblastoid cell lines (LCLs) obtained from each haplotyped patient. The effect of CB2 agonists, antagonists, and genetic deficiency on immune and acute murine colitis was assessed by measures of clinical disease activity and histologic inflammation.

A constructed CNR2 SNP HapMap revealed two discrete haplotype blocks. In non-Jews, select haplotypes were positively associated with the expression of anti-microbial antibodies, while others were negatively associated with these serotypes. Similarly, several haplotypes were associated with lower titers of antibodies. There was no significant association between CNR2 haplotype and levels of CB2 protein expression or receptor signaling. However, in mouse models of immune and acute colitis, CB2 agonists and antagonists strongly modulated intestinal inflammation.

Candidate gene analysis indicates that CNR2 is an IBD-associated gene, and stratifies patients with IBD in relation to their responses to anti-microbial peptides. Furthermore, mouse modeling indicated that CB2 strongly affected intestinal inflammation. These results show the endocannabinoid system plays a role in IBD disease susceptibility.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Ziring D, Wei B, Velazquez P, Schrage M, Buckley N E, Braun J. Formation of B and T cell subsets require the cannabinoid receptor CB2. Immunogenetics 2006; 58(9):714-25.
2. Sipe J C, Arbour N, Gerber A, Beutler E. Reduced endocannabinoid immune modulation by a common cannabinoid 2 (CB2) receptor gene polymorphism: possible risk for autoimmune disorders. J LeukocBiol 2005; 78(1):231-8.
3. Rousseaux C, Thum X, Gelot A, et al. Lactobacillus acidophilus modulates intestinal pain and induces opioid and cannabinoid receptors. NatMed 2007; 13(1):35-7.
4. Steffens S, Veillard N R, Arnaud C, et al. Low dose oral cannabinoid therapy reduces progression of atherosclerosis in mice. Nature 2005; 434:782-6.
5. Massa F, Marsicano G, Hermann H, et al. The endogenous cannabinoid system protects against colonic inflammation. JClinInvest 2004; 113(8):1202-9.
6. Kimball E S, Schneider C R, Wallace N H, Hornby P J. Agonists of Cannabinoid Receptor 1 and 2 Inhibit Experimental Colitis Induced by Oil of Mustard and by Dextran Sulfate Sodium. AmJPhysiol GastrointestLiver Physiol 2006.
7. Mathison R, Ho W, Pittman Q J, Davison J S, Sharkey K A. Effects of cannabinoid receptor-2 activation on accelerated gastrointestinal transit in lipopolysaccharide-treated rats. Br J Pharmacol 2004; 142(8):1247-54.
8. Wright K, Rooney N, Feeney M, Tate J, Robertson D, Welham M, Ward S. Differential expression of cannabinoid receptors in the human colon: cannabinoids promote epithelial wound healing. Gastroenterology 2005; 129(2):437-53.
9. Miller G, Lipman M. Release of infectious Epstein-Barr virus by transformed marmoset leukocytes. Proc Natl Acad Sci USA 1973; 70(1):190-4.
10. Anderson M A, Gusella J F. Use of cyclosporin A in establishing Epstein-Barr virus-transformed human lymphoblastoid cell lines. In Vitro 1984; 20(11):856-8.
11. Buckley N E, McCoy K L, Mezey E, Bonner T, Zimmer A, Felder C C, Glass M, Zimmer A. Immunomodulation by cannabinoids is absent in mice deficient for the cannabinoid CB(2) receptor. Eur J Pharmacal 2000; 396(2-3):141-9,
12. Chuchawankul S, Shima M, Buckley N E, Hartmann C B, McCoy K L. Role of cannabinoid receptors in inhibiting macrophage costimulatory activity. IntImmunopharmacol 2004; 4:265-78.
13. Rudolph U, Finegold M J, Rich S S, Harriman G R, Srinivasan Y, Brabet P, Bradley A, Birnbaumer L. Gi2 alpha protein deficiency: a model of inflammatory bowel disease. Clinlmmunol 1995; 15:101 S-5S.
14. Aranda R, Sydora B C, McAllister P L, Binder S W, Yang H Y, Targan S R, Kronenberg M. Analysis of intestinal lymphocytes in mouse colitis mediated by transfer of CD4+, CD45RBhigh T cells to SCID recipients. J Immunol 1997; 158(7):3464-73.
15. Brewer S, McPherson M, Fujiwara D, et al. Molecular Imaging of Murine Intestinal Inflammation With 2-Deoxy-2-[(18)F]Fluoro-d-Glucose and Positron Emission Tomography. Gastroenterology 2008.
16. Bjursten M, Willen R, Hultgren Hornquist E. Transfer of colitis by Galphai2-deficient T lymphocytes: impact of subpopulations and tissue origin. Inflamm Bowel Dis 2005; 11(11):997-1005.
17. Wei B, Velazquez P, Turovskaya O, Spricher K, Aranda R, Kronenberg M, Birnbaumer L, Braun J. Mesenteric B cells centrally inhibit CD4+ T cell colitis through interaction with regulatory T cell subsets. Proc Natl Acad SciUS A 2005; 102:2010-5.
18. Papadakis K A, Yang H, Ippoliti A, et al. Anti-flagellin (CBir1) phenotypic and genetic Crohn's disease associations. Inflamm Bowel Dis 2007; 13(5):524-30.
19. Mow W S, Vasiliauskas E A, Lin Y C, et al. Association of antibody responses to microbial antigens and complications of small bowel Crohn's disease. Gastroenterology 2004; 126(2):414-24.
20. Dubinsky M C, Kugathasan S, Mei L, et al. Increased Immune Reactivity Predicts Aggressive Complicating Crohn's Disease in Children. Clin Gastroenterol Hepatol 2008.
21. Vandewalle-El Khoury P, Colombel J F, Joossens S, et al. Detection of antisynthetic mannoside antibodies (ASigmaMA) reveals heterogeneity in the ASCA response of Crohn's disease patients and contributes to differential diagnosis, stratification, and prediction. Am J Gastroenterol 2008; 103(4):949-57.
22. Mei L, Targan S R, Landers C J, et al. Familial expression of anti-Escherichia coli outer membrane porin C in relatives of patients with Crohn's disease. Gastroenterology 2006; 130(4):1078-85.
23. Dubinsky M C, Lin Y C, Dutridge D, et al. Serum immune responses predict rapid disease progression among children with Crohn's disease: immune responses predict disease progression. Am J Gastroenterol 2006; 101(2):360-7.
24. Shen C, Landers C J, Derkowski C, Elson C O, Targan S R. Enhanced CBir1-specific innate and adaptive immune responses in Crohn's disease. Inflamm Bowel Dis 2008; 14(12):1641-51.
25. Taylor K, Targan S R, Mei L. High frequency IL23R haplotypes explain a high percentage of CD risk. DDW 2007, M2073.
26. Devlin S M, Yang H, Ippoliti A, et al. NOD2 variants and antibody response to microbial antigens in Crohn's disease patients and their unaffected relatives. Gastroenterology 2007; 132(2):576-86.
27. Takedatsu H, Taylor K D, Mei L, et al. Linkage of CD-related serological phenotypes: NFKB1 haplotypes are associated with anti-CBir1 & ASCA, and show reduced NF-kB activation. Gut 2008.
28. McGovern D P, Taylor K D, Landers C. et al. MAGI2 genetic variation and inflammatory bowel disease. Inflamm Bowel Dis 2008.
29. Chan M A, Stein L D, Dosch H M, Sigal N H. Heterogeneity of EBV-transformable human B lymphocyte populations. J Immunol 1986; 136(1):106-12.
30. Casali P, Notkins A L. Probing the human B-cell repertoire with EBV: polyreactive antibodies and CD5+ B lymphocytes. Annu Rev Immunol 1989; 7:513-35.
31. Klein T W. Cannabinoid-based drugs as anti-inflammatory therapeutics. Nat Rev Immunol 2005; 5(5):400-11.
32. Maestroni G J. The endogenous cannabinoid 2-arachidonoyl glycerol as in vivo chemoattractant for dendritic cells and adjuvant for Th1 response to a soluble protein. Faseb J 2004; 18(15):1914-6.
33. He J, Gurunathan S, Iwasaki A, Ash-Shaheed B, Kelsall B L. Primary role for Gi protein signaling in the regulation of interleukin 12 production and the induction of T helper cell type 1 responses. J ExpMed 2000; 191:1605-10.
34. Yuan M, Kiertscher S M, Cheng Q, Zoumalan R, Tashkin D P, Roth M D. Delta 9-Tetrahydrocannabinol regulates Th1/Th2 cytokine balance in activated human T cells. J Neuroimmunol 2002; 133(1-2):124-31.
35. Klein T W, Newton C A, Nakachi N, Friedman H. Delta 9-tetrahydrocannabinol treatment suppresses immunity and early IFN-gamma, IL-12, and IL-12 receptor beta 2 responses to Legionella pneumophila infection. J Immunol 2000; 164(12):6461-6.
36. Agudelo M, Newton C, Widen R, Sherwood T, Nong L, Friedman H, Klein T W. Cannabinoid receptor 2 (CB2) mediates immunoglobulin class switching from IgM to IgE in cultures of murine-purified B lymphocytes. J Neuroimmune Pharmacol 2008; 3(1):35-42.
37, Matias 1, Pochard P, Orlando P, Salzet M, Pestel J, Di Marzo V. Presence and regulation of the endocannabinoid system in human dendritic cells. Eur J Biochem 2002; 269(15):3771-8.
38. Blumberg R S, Saubermann L J, Strober W. Animal models of mucosal inflammation and their relation to human inflammatory bowel disease. Curr OpinImmunol 1999; 11(6):648-56.
39. Rakoff-Nahoum S, Hao L, Medzhitov R. Role of toll-like receptors in spontaneous commensal-dependent colitis. Immunity 2006; 25(2):319-29.
40. Araki A, Kanai T, Ishikura T, et al. MyD88-deficient mice develop severe intestinal inflammation in dextran sodium sulfate colitis. JGastroenterol 2005; 40(1):16-23,
41. Ogawa A, Andoh A, Araki Y, Bamba T, Fujiyama Y. Neutralization of interleukin-17 aggravates dextran sulfate sodium-induced colitis in mice. Clinlmmunol 2004; 110(1):55-62.

1. A method of diagnosing a Crohn's Disease subtype in an individual, comprising:
determining the presence or absence of a risk haplotype at the CNR2 genetic locus; and
determining the presence or absence of a high expression of ASCA antibody relative to a normal subject,
wherein the presence of the risk haplotype at the CNR2 genetic locus and the presence of the high expression of ASCA antibody relative to a normal subject in the individual is indicative of the Crohn's Disease subtype.

2. The method of claim 1, wherein the risk haplotype at the CNR2 genetic locus comprises Block 1 Haplotype 1.

3. The method of claim 1, wherein the risk haplotype at the CNR2 genetic locus comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, or a combination thereof.

4. The method of claim 1, wherein the high expression of ASCA antibody relative to a normal subject comprises a median antibody titer level above 0.1.

5. The method of claim 1, wherein the individual is non-Jewish.

6. A method of diagnosing a Crohn's Disease subtype in an individual, comprising:
determining the presence or absence of a Block 1 Haplotype 2 at the CNR2 genetic locus; and
determining the presence or absence of a low expression of ASCA and/or Cbir1 antibodies relative to a normal subject,
wherein the presence of the Block 1 Haplotype 2 at the CNR2 genetic locus and the presence of the low expression of ASCA and/or Cbir1 antibodies relative to a normal subject in the individual is indicative of the Crohn's Disease subtype.

7. The method of claim 6, wherein the low expression of ASCA antibody relative to a normal subject comprises a median antibody titer level of less than 0.5.

8. The method of claim 6, wherein Block 1 Haplotype 2 at the CNR2 genetic locus comprises SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, or a combination thereof.

9. The method of claim 6, wherein the individual is non-Jewish.

10. A method of diagnosing a Crohn's Disease subtype in an individual, comprising:
determining the presence or absence of a Block 2 Haplotype 3 at the CNR2 genetic locus; and
determining the presence or absence of a low expression of OmpC and/or I2 antibodies relative to a normal subject,
wherein the presence of the Block 2 Haplotype 3 at the CNR2 genetic locus and the presence of a low expression of OmpC and/or I2 antibodies relative to a normal subject is indicative of the Crohn's Disease subtype in the individual.

11. The method of claim 10, wherein the low expression of OmpC antibody relative to a normal subject comprises a median antibody titer level of less than 18.5.

12. The method of claim 10, wherein the low expression of I2 antibody relative to a normal subject comprises a median antibody titer level of less than 26.0.

13. The method of claim 10, wherein the Block 2 Haplotype 3 at the CNR2 genetic locus comprises SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, or a combination thereof.

14. A method of defining a CNR2 signaling phenotype in an individual with Crohn's Disease, comprising:
determining the presence or absence of one or more risk haplotypes at the CNR2 locus in the individual;
determining the presence or absence of one or more risk serological markers in the individual; and
defining the CNR2 signaling phenotype based upon the presence of one or more risk haplotypes at the CNR2 locus and the presence of one or more risk serological markers in the individual.

15. The method of claim 14, wherein the one or more risk haplotypes at the CNR2 locus comprise Block 2 Haplotype 3.

16. The method of claim 14, wherein the one or more risk serological markers comprises OmpC and/or I2 antibodies.

17. The method of claim 14, wherein the CNR2 signaling phenotype is characterized by approximately half the level of CB2 signaling relative to levels found in a healthy subject.

18. The method of claim 14, wherein the one or more risk haplotypes at the CNR2 locus comprise Block 1 Haplotype 1 and/or Block 1 Haplotype 2.

19. The method of claim 14, wherein the one or more risk serological markers comprise ASCA and/or Cbir antibodies.

20. The method of claim 14, wherein the individual is non-Jewish.

21. A method of treating Crohn's Disease in an individual, comprising:
determining hypofunctioning CB2 signaling based upon the presence of Block 2 Haplotype 3, OmpC antibodies, and/or I2 antibodies in a sample taken from the individual; and
treating the Crohn's Disease in the individual.

22. A method of diagnosing susceptibility to Crohn's Disease in an individual, comprising:
determining the presence or absence of a risk haplotype at the CNR2 genetic locus; and
determining the presence or absence of a risk serological marker,
wherein the presence of the risk haplotype at the CNR2 genetic locus and the presence of the risk serological marker in the individual is indicative of susceptibility to Crohn's Disease.

23. The method of claim 22, wherein the risk serological marker comprises ASCA and/or Cbir antibodies.

24. The method of claim 22, wherein the risk serological marker comprises OmpC and/or I2 antibodies.


SEQUENCE LISTING (18 sequences)

SEQ ID NO: 1 - DNA, 801 bp, Homo sapiens
tgagttaactttgcagcaatctgatgctacagatagctaatattatccctaatgagcaataatcaagcagaatttgactgaactaggacaaaaggttagtggttaagagggcaggctttgaagtcaaacagaactaggttggagacctcagctctggtacttagcagggtgaccttagacaagtgactttgcatctttgaggttgccacatctgtaaaatgaagataactaagaccttcttattaggttgttgggaaacatgaagtgaaataaggcatttaaaaacacttagccccctgcctggcacatagtaaaggctcaataaaagtgagccggcacatggtaggtctatcctaccgtttattgagttaactcaattaatccctattacatgacatgamcctgcatatttcctttgttgtactgcctgtaattatcttctttattgacttattggctgtcttgcaagcaggatatagactgtaagccccatgagacaggggcacagtctcatttatggctgggtactcaggaccctgcacagagcctggctcatgtttatgagatgaatgaataagtgaatagctagtggatattttgtcttgaaagtattaaacgaagacatgtttaaccaatatctttcattttattccatttgaggtcccaaataattgcattagaattcctctgcaatcttcctgccgccaacaatttctcttgttggctgttttaggctcctctgggaggaacccagcctccctcatatgaaaaatgagtaccctgggatctattttattttat

SEQ ID NO: 2 - DNA, 801 bp, Homo sapiens
ctgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaggcctgggacaggstcagtacaagtcaggtcaggcttcatgcctgcatcctccagagaccacaggagccaaagcgagcctccaggcccagcaatgagggacttgggagaaatctgagaagaatgggttgttctcttgggaagtcagggtatcagatgggatggacatccaggtcttctctctgcctaattgtcaaggcctccttggctctggagctatgaaaggccccactttcaagtcacccttgccactgaggaccgaggactatgctatgatgaggattaaggtgttgacttgcctctttcagagataaatgacaagccttcagtttggggcatcctgttgtttgggcgaggaccctctctgatattctgataatccccacctgtaacctcaggtccctggaaatacagag

SEQ ID NO: 3 - DNA, 801 bp, Homo sapiens
gccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagrcacctagacacggacccctttttgctgatgagtgttgggactgactcctggaagacagcctggccttgcccacctgcacacagtctgttggataggtagggccacgaggagtagccaggtaggcgagacacaaaaggcctgggacagggtcagtacaagtcaggtcaggcttcatgcctgcatcctccagagaccacaggagccaaagcgagcctccaggcccagcaatgagggacttgggagaaatctgagaagaatgggttgttctcttgggaagtcagggtatcagatgggatggacatccaggtcttctctctgcctaattgtcaaggcctccttggctctggagctatgaaaggccccactttcaagtcacccttgccactgaggaccgaggact

SEQ ID NO: 4 - DNA, 801 bp, Homo sapiens
ggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcarttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaggcctgggacagggtcagtacaagtcaggtcaggcttcatgcctgcatcctccagagaccacaggagccaaagcgagcctccaggcccagcaatgagggacttgggagaaatctgagaagaatgggttgttctcttgggaagtcagggtatcagatgggatggacatccaggtcttctctctgcctaattgtcaaggcctcct

SEQ ID NO: 5 - DNA, 801 bp, Homo sapiens
tcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcacsgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaggcctgggacagggtcagtacaagtcaggtcaggcttcatgcctgcatcctccagagaccacaggagccaaagcgagcctccaggcccagca

SEQ ID NO: 6 - DNA, 801 bp, Homo sapiens
tttcccactgatccccaatgactacctgctgagctggctcctgttcatcgccttcctcttttccggaatcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctyactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaggcctgggacagggtcagtacaag

SEQ ID NO: 7 - DNA, 801 bp, Homo sapiens
gggcactggtgaccctgggcatcatgtgggtcctctcagcactagtctcctacctgcccctcatgggatggacttgctgtcccaggccctgctctgagcttttcccactgatccccaatgactacctgctgagctggctcctgttcatcgccttcctcttttccggaatcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgcyttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagc

SEQ ID NO: 8 - DNA, 801 bp, Homo sapiens
cccctctctctcttccagggagtgaactgatttctgacttgagttgtttaaattgggaagaggcctcatcagcaatcagagaggtctagatctctggaatctggccacggagtgattttcccatcagcctctgtctcggtgactgaggatctcggggcttcttcttttgcctctgacccaaggcccctcacacacttcttccagtgagccaggcagtgatgggcagaggagcggatctctccactccgtagagcatagatgacagggttgaccatggagttgatgaggcacagcatggagcagaaagcaaaggccttcttgacctggtcactgagcgtagtggccaggctgtgggccatgagggccagcactgggaaccaacagatgaggagcacagccarcactagccctagggtcttggccaacctcacatccagcctcattcgggccattcctggcacctgcctgtcctggtggccagacaagctggccacatgctgatgggccttccagagaacatgcccataggtgtagatgattccggaaaagaggaaggcgatgaacaggagccagctcagcaggtagtcattggggatcagtgggaaaagctcagagcagggcctgggacagcaagtccatcccatgaggggcaggtaggagactagtgctgagaggacccacatgatgcccagggtcaccagtgcccttccacgggtgagcagagctttgtaggaaggtggatagcgcaggcagaggtatcggtcaatggcggtcagcaggaggctacccacagaggctgtg

SEQ ID NO: 9 - DNA, 801 bp, Homo sapiens
ggcacagcatggagcagaaagcaaaggccttcttgacctggtcactgagcgtagtggccaggctgtgggccatgagggccagcactgggaaccaacagatgaggagcacagccaacactagccctagggtcttggccaacctcacatccagcctcattcgggccattcctggcacctgcctgtcctggtggccagacaagctggccacatgctgatgggccttccagagaacatgcccataggtgtagatgattccggaaaagaggaaggcgatgaacaggagccagctcagcaggtagtcattggggatcagtgggaaaagctcagagcagggcctgggacagcaagtccatcccatgaggggcaggtaggagactagtgctgagaggacccacatgatrcccagggtcaccagtgcccttccacgggtgagcagagctttgtaggaaggtggatagcgcaggcagaggtatcggtcaatggcggtcagcaggaggctacccacagaggctgtgaaggtcatagtcacgctgccaatcttcagcaggaagacagccttggaatccacaccatggaaaacatggaaattcacaaagctgcatgcaaagaccacactggccaggaagtcagccccagccaagctgccaatgaacaggtatgagggcttccggcggagttggtgggaggacaggatcagatagagcacagccacgttctccagggcacttagcaggcccagaagagtgcacaacacagcaacagctgtcttctggggaccactcaggatcatgtaatccttca

SEQ ID NO: 10 - DNA, 201 bp, Homo sapiens
tatcagtgtgccagcagagttctgaatattaagaatcaccgatgcatgcaaaaacattttgtgacctgatgctgtacagatgtaagagtaaaagagaagcrggagtctttctgaacctattctgggctgcctgattaaaaaagaaaaaaggccaggcgcggtggctcacgcctgtaatcccagcactttgggaggccgagg

SEQ ID NO: 11 - DNA, 701 bp, Homo sapiens
tggtgttaggggctgcagcctacagcctggttcatactgagcatctgggaagggaaacttgggacctgagaacctgctcagaaaaggtgtgcctgggggtttttctaacctgaagtgaaagcgtgaagtgaaagaagccacagtagcggctggtcccacgttgctgctgggtttaaatctctcagagctggaggtaaggargagaatgataccctgcctgtggctaccacacctacagtgggaagctgagatttgatgccacaagcctcaccgtttttatccccaactgggagtagggaagcctaggttctagtccttgtcctgctactgagtgactttaggcaggtcccttttcctatctaaggcctttcttttctttctttgatttctattaaaaaaatatagatagtgtcttgctatattgctcaggctggtctcaaactcctggcctcaagcagtccttccacctcagcctcccaaaatgttgggattatagacatgagccaccttgcctgaccatccgggcctttcttttctcatcttcaaagcagttggattgatggaggtgattcccaaggtctcctcgaggaaacttagcatgttggatcacaatgtcacaggtttgggaaccagaccttcctggattaaaattcagctctgcaactttcaggatgtgtgaccttgggcaccttactgcacct

SEQ ID NO: 12 - DNA, 1000 bp, Homo sapiens
cagattaatgatacaagtttaccaaattcatgtcactgtacctggtttacagtaggtatctaatcaaaggcaggagatgcttaatagagttagtttatagtgttcttacaggattctgtgccaggcatgggggctctgcctgtaatcccagcactttgggaggcagagacaggtggattatttgagcccaggaatagcctggccaacatggtaaaaccccatctctacaaaaaatacaaaaattagctgggcatggtgcatgtatgtagtcccagctactggggagactgaggcaggagaatcacttgagcccaggaagtcacggctgcagtaagcagtgattgttccactgtgctctagcccgggctacagagtgagaccttgtctcaaaaaaaaaaaaaaaagaaaagaaaagaaaagaagaagaagaaaaggattctgtcctccagctatgcctgaccttcgcctgagagccaaggcagatagaacaatccctacttmctctcagctgacactttgccaccactgaggacaaaccaccacattttggaaaacttgttcaacctgcaagaatgacccatcagaaacagacccgctgggcgaggtggctcacgcttataatcccagcactttgggaggccgaggcaggcagatcacctgaggtccggagtttgagaccagactggccaacatggtgaaaccccatctctactaaaaatacaaaattagccgggcgtggtggtgcactcttgtaatcccagctactcgggaggctgaggcaagagaattgcttgaatccgggaggcgaagtttgcagtgagccgagatcgcgccactgcactccagcctgggcaacaagagtgagactctgtctcaaaaaaaaaaaaaagaaatagatcaaagtgaccctcttgacctgctgcccagctggctgggcccctgctgcagtgttcagccatggggctttccttccacgtttcttctcttagtccccctctgg

SEQ ID NO: 13 - DNA, 824 bp, Homo sapiens
tgtgtgtgtgtgtgtgtgtgagcctatgaattatactgaaaatccaaatcaggccaggcacggtggctcacacctataatcccagcactttgggaggctaaggcaggcagatcacctgagttcaggagttcgagaccagcctggccaacatggtgaaaccccacctctactaaaaatacaaaaattggccaagtgtggtggtgggtgcctgtaatcccagctacttgggaggctgaggcaggagaattgcttgaatctgggaggcagaggttgcagtgagccgagatcatgccattgcactccagcctgggcaacaagagcaaaactccatctcaaaaaaaaaaaaaaaaaaagcccaaatcttacctctttttataactactttgatagcaaaacccacctcctccaagaagcattctctggtctccctttattgagtcttactgcacagaccactgtctaccttgtattatatgtgtccattccacatcttgcttcctrgattgtgagctccttgtcttgtgagtaatatctgggtctgggagtggagtcatctttgtatctcacttgctcaaaaaagtgtctcctagcacaatggtatccccacagtagactcttggtggaggtcttttgatagcagccagagctgagcaaaacagtcttggggagatggaactcagcccagccaggcaaggcaatttgatggcctcaagctcccactcctcaatcccaagcccatgacagacacttctcaccactccaagtccttgtgcctcctgagcctgaccatatttctcaccctagtgttcctggcccggagaacc

SEQ ID NO: 14 - DNA, 801 bp, Homo sapiens
cccaaatcttacctctttttataactactttgatagcaaaacccacctcctccaagaagcattctctggtctccctttattgagtcttactgcacagaccactgtctaccttgtattatatgtgtccattccacatcttgcttcctggattgtgagctccttgtcttgtgagtaatatctgggtctgggagtggagtcatctttgtatctcacttgctcaaaaaagtgtctcctagcacaatggtatccccacagtagactcttggtggaggtcttttgatagcagccagagctgagcaaaacagtcttggggagatggaactcagcccagccaggcaaggcaatttgatggcctcaagctcccactcctcaatcccaagcccatgacagacacttctcaycactccaagtccttgtgcctcctgagcctgaccatatttctcaccctagtgttcctggcccggagaaccagacttgcagaaagaggttggaaccacaagcagcagccctgaagcagagtgtgaactagaaggatggtgcctgcccaggctcttgaccttgtgaacaggccaccaggacagtcactcctaaaatgggtttggcagtggaggtgggacagcactgggcagggaaggaacttaaaatctctctagtgatataggtcccaggggaggtttattgatataaggcaaaatatggaagctttcctagaggagagatgaaaagggctggcaaaggagttgtttcctgagtcaattcctttaataaattcctcctggggtttccagcacctcaactggc

SEQ ID NO: 15 - DNA, 782 bp, Homo sapiens
ctgagtcaattcctttaataaattcctcctggggtttccagcacctcaactggcatgaagcctggcacaaagtaagcatctaagtatttaaaatggatttatagaatcaatgaatgtctattaggagaaaagcagccttcttcagacccacttctgtgcttctttttcccttggctgccagcaagcccagagctcaatgcagtgtggaagtgcagtcacagggccattcaaggtcggttcttgatttatttattgctccttttagtggtttctaatgccttkcttttattttaactgccccaatttataatgtagccacctctactctttttaaggaaagaatgtgtaggctgggggtggtggctcatgctgtaatcccagcactttgggaggccgaggcaggcggatcatttgaggtcaggagttagaaaccagcctggccaacatggtgaaaccctgtctctactaaaactacaaaaattagccaggagtagtggtgggtgcctgtaatcccagctacttgggaggctgaggcaggagaatcacttgagcctgtgaggtggaggttgcagcgagcagagatcgcaccattgcacttcagcctgggcgacagagcgagactctgtctcaaaaaaataaaaggaaagaatgtgtaatgtataggttaagagcaaaggattgttattaaggtcttctatctatagtccctttctctaatgtctacgggttatcaaagaaactcagtcttccctgtggggccgaaggattccatccccagcctg

SEQ ID NO: 16 - DNA, 701 bp, Homo sapiens
cgggttatcaaagaaactcagtcttccctgtggggccgaaggattccatccccagcctgctgtccactcaccattcatcacccccacccaggctgtctggattcactcagtctccccagacctttcctggaccttccttctgcatcatcatctccagcctctctcctgggcagagcagagctaaatgatgtctgaggcctytttccactttagtcaaggttaagtgccaaggctttagaaggaaacatcactgagttcaaatctcaggtcggccactgataggctgtgtgaccttggggaaagttactcaaactctctgagcttcagtttcttcctctgtgaatggggaaactaatccctatgttctgtatgtgcagattaaataattatttaaaactaattaagcaggagattggttgcctgattgaactatcaagagtgttggatgtggtggaaagaaaacagtcctgagttcggaatctcaggtgctcagggtttgagccctggctctggcaccagctggatctatccagaagccacctactctctcttgtcccagtattgccatttgtaaagaagattgggggataacgatgagtgcttggcactgtgctagcactttctacacattatctcatttaaatctcaaaacaacccttcaagaatcttattattttctgtattttatagatgaagaaatt

SEQ ID NO: 17 - DNA, 1617 bp, Homo sapiens
gtctggattcactcagtctccccagacctttcctggaccttccttctgcatcatcatctccagcctctctcctgggcagagcagagctaaatgatgtctgaggcctctttccactttagtcaaggttaagtgccaaggctttagaaggaaacatcactgagttcaaatctcaggtcggccactgataggctgtgtgaccttggggaaagttactcaaactctctgagcttcagtttcttcctctgtgaatggggaaactaatccctatgttctgtatgtgcagattaaataattatttaaaactaattaagcaggagattggttgcctgattgaactatcaagagtgttggatgtggtggaaagaaaacrgtcctgagttcggaatctcaggtgctcagggtttgagccctggctctggcaccagctggatctatccagaagccacctactctctcttgtcccagtattgccatttgtaaagaagattgggggataacgatgagtgcttggcactgtgctagcactttctacacattatctcatttaaatctcaaaacaacccttcaagaatcttattattttctgtattttatagatgaagaaattggggctcaggaatgttaagaaactttctgggggctgggtaccatggctcatacctgtagtcccagcactttgggaggctgaggtgggcagatcacttgagcccagggcttagagaccagcccgggtaacatgaaactccatctctacaaaaaatacaaaaattagctgggcatggtgttgtgtgcctgtagtcccagccactcaggaagctgaggtggtggattgctggagcccaggagagtgaagctgcagtgagccgtgatcacaccactgcactctagcctgagtgacagagcagaccctgtctcaaattaaaaaaaaaaaaagggactgggcacagtggctcatgcctgtaatcccaatactttgggaggccgaggtgggtggatcgccagaggtcaggagttcaagaccagcctgaccaacatggtgaaacttcatctctacagtaaaaaatacaaaagtaactgggcgttgtggctcgcctgtaatcctaactactcgggtggctgaggcaggagaatcacttgaacttgggaggtggaggttgcactgagctgagatcatgccattgcactgcagcctgggcaacaaaagtgaaactctgtctcaaaaaaaaaaaatttttttaataaaaggaaggaaggaaggaaggaagcaaggaaggaaggaagctaggaaggaaggaaggaagggagggaaagaaagttggccaggcacagtggctcacacctgtaatcccagcactttgggaggccaagatggtggagcacttgaggtcaggagttcgagaccagcctggccaacaccgtgaaaccccatctgtattataaataataataataaaaaaatcagccaggcaggagaatcacttgaacctgggaagcagaggctgcagtgagccacgatcctgccactgcactccaccctgggcaacagagtgaaattttgtctcaaaaaaaaaaagaaagaaagaaagaaaaagaaacttgctggggacattgggaatc

SEQ ID NO: 18 - DNA, 1776 bp, Homo sapiens
caggtcctgggagaggacagaaaacaactggactcctcagcccccggcagctcccagtgcccagccacccacaacacaacccaaagccttctagacaagctcagtggaatctgaagggcccaccccatggaggaatgctgggtgacagagatagccaatggctccaaggatggcttggattccaaccctatgaaggattacatgatcctgagtggtccccagaagacagctgttgctgtgttgtgcactcttctgggcctgctaagtgccctggagaacgtggctgtgctctatctgatcctgtcctcccaccaactccgccggaagccctcatacctgttcattggcagcttggctggggctgacttcctggccagtgtggtctttgcatgcagctttgtgaatttccatgttttccatggtgtggattccaaggctgtcttcctgctgaagattggcagcgtgactatgaccttcacagcctctgtgggtagcctcctgctgaccgccattgaccgatacctctgcctgcgctatccaccttcctacaaagctctgctcacccgtggaagggcactggtgaccctgggcatcatgtgggtcctctcagcactagtctcctacctgcccctcatgggatggacttgctgtcccaggccctgctctgagcttttcccactgatccccaatgactacctgctgagctggctcctgttcatcgccttcctcttttccggaatcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaaggcctgggacagggtcagtacaagtcaggacaggcttcatgcctgcatcctccagagaccaccaggagccaaagcgagcctccaggcccagcaatgagggacttgggagaaatctgagaagaatgggttgttctcttgggaagtcagggtatcagatgggatggacatccaggtcttctctctgcctaattgtcaaggcctccttggctctggagctatgaaaggccccactttcaagtcacccttgccactgaggaccgaggactatgctatgatgaggattaaggtgttgacttgcctctttcagagataaatgacaagccttca